(19) United States
(12) Patent Application Publication
(10) Pub. No.: US 2020/0306155 A1
(43) Pub. Date: Oct. 1, 2020

(54) Method Of Making A Color Cosmetic Composition Containing Wax Blend

(71) Applicant: ELC MANAGEMENT LLC, Melville, NY (US)

(72) Inventor: Carla Bekdach, Beirut (LB)

(21) Appl. No.: 16/368,123

(22) Filed: Mar. 28, 2019

(51) Int. Cl. A61K 8/31; A61K 8/92; A61K 8/26; A61K 8/891; A61Q 1/06; A61Q 1/08; A61Q 1/10; A61Q 1/12

(52) U.S. Cl. CPC A61K 8/31; A61K 8/927; A61K 8/26; A61K 8/891; A61Q 1/06; A61Q 1/08; A61K 2800/805; A61Q 1/12; A61K 2800/31; A61K 2800/592; A61K 2800/43; A61K 2800/10; A61Q 1/10

(57) ABSTRACT

The present invention relates to a method for forming an anhydrous, moldable, cosmetic composition. According to the method, four cosmetic waxes are combined in a vessel to form a first phase. A solvent and wetting agent are combined in a separate vessel to form a second phase. Then, the first and second phases are combined to form a cosmetic composition. The cosmetic composition has a high pigment load while having excellent feel and payoff characteristics.

### FIELD OF THE INVENTION

The present invention relates to a method for forming an anhydrous, moldable, cosmetic composition comprising a blend of waxes.

### BACKGROUND OF THE INVENTION

In recent years, there has been a strong trend toward the use of silicone fluids in makeup compositions. A major reason for their popularity is the elegant feel provided by the silicones: the product containing them goes onto the skin smoothly, with an excellent slip, and yet does not produce the greasy, heavy feel that non-silicone oils frequently leave. Silicone oils are now common components of virtually all types of makeup compositions, both liquid and powder, for example, foundations, concealers, eyeshadows and eyeliners, lipsticks and lip pencils, and blushes. Another attractive aspect of the silicone oils is their tendency to produce a very shiny appearance on the skin to which they are applied. This is often desirable for certain types of cosmetics, particularly for those aimed at a younger consumer, to whom a glowing or glossy appearance is very appealing, or for cosmetics intended for evening wear, where subdued lighting permits a greater latitude in the shine produced by the cosmetic.

However, for certain types of cosmetic products, and/or for certain types of consumers, a significant amount of shininess is not desired and may even be inappropriate. A more mature user may not be flattered by a very glossy or shiny makeup. The fine lines and wrinkles which characterize a more mature skin are emphasized by a glossy product which ends to directly reflect light. More preferable for the consumer of a certain age is a makeup which will scatter or diffuse light, thereby providing a “soft focus”, which blurs lines and hides blemishes. Attempts to achieve this type of masking in the past have largely relied on the use of higher levels of pigment, but this frequently results in a heavy, cakey product which does not flatter a majority of consumers.

Similarly, it may also simply be desirable to eliminate shininess for products to be used in certain environments. For example, glossy makeup is often perceived as inappropriate in a conservative office environment, and/or may be too glaring under the harsh lights of the typical office. In these circumstances, a more matte finish to the cosmetic product may be called for. Traditionally, this has been achieved by the addition of solid powders, such as mica, silica, talc, and the like, to the formulation. In the case of a silicone oil-based composition, however, counteracting the shine produced tends to be more difficult than with more traditional cosmetic oils, thus requiring addition of even greater amounts of the solid fillers. A larger proportion of solids in a formulation, however, results in a heavy, draggy feel on the skin, thereby canceling out to some extent some of the benefit of the silicone oils. Alternately, a more matte appearance can be achieved by including one or more volatile components, such as water or volatile oils, in the formulation; when the volatile evaporates upon application, this increases the concentration of pigment relative to the composition as a whole, resulting in a more matte look. However, this can also result in a dry, cakey look on the skin. Moreover, the use of volatiles, particularly water, is not appropriate and/or feasible in all products, such as anhydrous lipstick products, and also results in the necessity of finding specialized, and often expensive, airtight packaging to prevent loss of the volatile from the product in the package. results in the necessity of finding specialized, and often expensive, airtight packaging to prevent loss of the volatile from the product in the package.

Based on the foregoing, there is an ongoing need for a cosmetic composition which can deliver excellent payoff and aesthetic effects without the drawbacks of the unpleasant feel exhibited by existing formulations when high levels of solids are included.

### SUMMARY OF THE INVENTION

The present invention relates to a method for forming an anhydrous, moldable, cosmetic composition. According to the method, four cosmetic waxes are combined in a vessel to form a first phase. A solvent and wetting agent are combined in a separate vessel to form a second phase. Then, the first and second phases are combined to form a cosmetic composition. The cosmetic composition has a high pigment load while having excellent feel and payoff characteristics.

The cosmetic composition may be in the form of an eyeshadow, blush, face powder, lipstick, and the like.

This, and other aspects of the present invention are described hereinafter.

### DETAILED DESCRIPTION OF THE INVENTION

While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level, and therefore they do not include solvents or by-products that may be included in commercially available materials, unless otherwise specified. The term “weight percent” may be denoted as “wt. %” herein.

All molecular weights, as used herein, are weight average molecular weights expressed as grams/mole, unless otherwise specified.

All ratios are weight ratios unless specifically stated otherwise.

Herein, “anhydrous” means that a composition of ingredients contains little or no water. Preferably, anhydrous systems, compositions, or materials contain no water.

Herein “hot pour” means a cosmetic formulation that must be heated to melt and disperse its constituent ingredients which then solidify upon cooling.

Herein, “cs” means centistoke.

The term, “moldable”, as used herein, means that a material is capable of being molded or shaped, at room temperature, without cracking or breaking.

Herein, “molecular weight” is measured in terms of the weight average molecular weight and is measured by gel permeation chromatography (GPC).

The term, “nonvolatile solvent”, as used herein means a solvent which does not quickly evaporate at room temperature. Preferably, a nonvolatile solvent will have an evaporation rate of about 0.002 mg/cm²/min or less. The evaporation rates herein are expressed in mg of solvent evaporated per unit area (cm²) per unit time (minute).

The term, “volatile solvent”, as used herein, means a solvent which quickly evaporates at room temperature. Preferably, a volatile solvent will have a rate of about 0.01 mg/cm²/min or more.

The term, “water-soluble”, as used herein, means that the polymer is soluble in water. In general, the polymer should be soluble at 25° C. at a concentration of at least 0.1 wt. % of the water solvent, preferably at least 1 wt. %, more preferably at least 5 wt. %, most preferably at least 15 wt. %.

The term “water-insoluble,” as used herein, means that a compound is not soluble in water. Thus, the compound is not miscible with water.

### Wax Blend

The present compositions include a blend of at least four cosmetically acceptable waxes. Each wax component is characterized by its specified melting temperature range or melting behavior. The waxes may be natural or synthetic. While each of the at least four wax components is different from one another, it may be possible for melting temperature ranges of the waxes to overlap. It is believed that the blend of waxes, according to the specified melting characteristics, provides a moldable cosmetic composition, with a high pigment load, and desirable payoff and drag characteristics.

The total amount of the wax blend which may be present in the compositions may be from about 2 wt. % to about 20 wt. %, more preferably from about 5 wt. % to about 15 wt. %, and most preferably from about 8 wt. % to about 12 wt. % of the composition.

### First Wax Component

The first wax component is characterized by having a melting temperature of from about 60° C. to about 65° C. Nonlimiting examples of suitable first wax components include, for example, paraffin wax, petrolatum, ceresin wax and the like. Particularly preferred is synthetic beeswax. The first wax component may be present in the compositions herein at a level of from about 1 wt. % to about 10 wt. %, more preferably from about 3 wt. % to about 8 wt. %, and even more preferably, from about 4 wt. % to about 6.5 wt. % of the composition.

### Second Wax Component

The second wax component should generally be capable of melting upon contact with skin. Therefore, the second wax has a melting temperature of from about 33° C. to about 46° C. Preferably, the second wax has a melting temperature of less than about 42° C. Exemplary waxes may include laurel wax, jojoba butter, microcrystalline wax such as Paramelt® LMP (available from Paramelt), and natural waxes such as myrica pubescens fruit cera wax. Particularly preferred is stearyl dimethicone/octadecene (available under the tradename Dowsil 2503® from Dow Corning). The second wax component may be present in the compositions at a level of from about 0.5 wt. % to about 5 wt. %, more preferably from about 1 wt. % to about 4 wt. %, and even more preferably, from about 1 wt. % to about 2 wt. % of the composition.

### Third Wax Component

The third wax component is characterized by having a melting temperature of from about 88° C. to about 96° C. A nonlimiting example of a suitable third wax components includes a microcrystalline wax, such as Microcrystalline Wax SP 88, available from Strahl & Pitsch. The third wax component may be present in the compositions herein at a level of from about 1 wt. % to about 5 wt. %, more preferably from about 2 wt. % to about 4 wt. %, and even more preferably, from about 2.5 wt. % to about 3 wt. % of the composition.

### Fourth Wax Component

The fourth wax component is characterized by having a melting temperature of from about 74° C. to about 99° C. Nonlimiting examples of suitable second wax components include a microcrystalline wax such as Multiwax W-835, available from Sonneborn. The fourth wax component may be present in the compositions herein at a level of from about 0.5 wt. % to about 5 wt. %, more preferably from about 1 wt. % to about 4 wt. %, and even more preferably, from about 1 wt. % to about 2 wt. % of the composition.

### Solvent

The compositions include at least one solvent. The solvent may be any lipophilic cosmetically acceptable solvent. Suitable exemplary solvent types may include silicone-based solvents, ester-based solvents, hydrocarbon-based solvents, and mixtures thereof. A silicone solvent can be a dimethicone, cyclomethicone or a single molecule silicone. An ester-based solvent can be defined as an organic compound made by replacing the hydrogen of an acid by an alkyl or other organic group. A hydrocarbon solvent is defined as any fluid consisting of saturated carbons.

The solvent may be selected from, for example, nonvolatile hydrocarbon-based oils, where appropriate fluorinated, and/or nonvolatile silicone oils.

Nonlimiting examples of suitable nonvolatile hydrocarbon-based oils include hydrocarbon-based oils of either animal or plant origin. Such oils may include, for example, triglycerides consisting of fatty acid esters of glycerol, fatty acids which may have chain lengths varying from C₄ to C₂₄, these chains being linear or branched, and saturated or unsaturated. Exemplary oils may include heptanoic or octanoic acid triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion-flower oil, musk rose oil and mixtures thereof. Other oils may include shea butter or caprylic/capric acid triglycerides, such as those sold under the names Miglyol 810®, 812® and 818® available from Dynamit Nobel.

According to one embodiment, the solvent may also comprise synthetic ethers containing from 10 to 40 carbon atoms, other than those corresponding to the definition of dry oils, such as diisocetyl ether.

In another embodiment, the solvent may comprise linear or branched hydrocarbons of mineral or synthetic origin, such as petroleum jelly, polydecenes, hydrogenated polyisobutene such as Parleam®, squalane and liquid paraffins, and mixtures thereof.

According to another embodiment, the nonvolatile solvent may be selected from synthetic esters, other than those corresponding to the definition of the dry oils, such as oils of formula R₁COOR₂ in which R₁ represents a linear or branched fatty acid residue containing from 1 to 40 carbon atoms and R₂ represents an especially branched hydrocarbon-based chain containing from 1 to 40 carbon atoms, on condition that R₁+R₂>10. Such oils may include, for example, purcellin oil (cetostearyl octanoate), C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalcohol heptanoates, octanoates, decanoates or ricinoleates such as propylene glycol dioctanoate, and mixtures thereof. Other such oils may include hydroxylated esters such as isostearyl lactate, diisostearyl malate and 2-octyldodecyl lactate, polyol esters, pentaerythritol esters, and mixtures thereof.

According to yet another embodiment, the nonvolatile solvent may be selected from fatty alcohols or higher fatty acids. Suitable fatty alcohols include those that are liquid at room temperature, with a branched and/or unsaturated carbon-based chain containing from 12 to 26 carbon atoms. Nonlimiting examples may include octyldodecanol, isostearyl alcohol, oleyl alcohol, 2-hexyldecanol, 2-butyloctanol and 2-undecylpentadecanol. Suitable higher fatty acids include, for example, oleic acid, linoleic acid or linolenic acid, and mixtures thereof.

The nonvolatile silicone oils that may be used in the present compositions may also be nonvolatile polydimethylsiloxanes (PDMSs), optionally including polydimethylsiloxanes comprising alkyl or alkoxy groups, that are pendent and/or at the end of a silicone chain, the groups each containing from 2 to 24 carbon atoms. Such PDMSs may include, for example, phenyl silicones, such as phenyl trimethicones, phenyl dimethicones, phenyltrimethyl-siloxydiphenylsiloxanes, diphenyl dimethicones, diphenylmethyldiphenyltrisiloxanes, and mixtures thereof.

According to a preferred embodiment, the solvent is a nonvolatile solvent selected from ethanol, octyldodecanol, isopropanol, and mixtures thereof. Octyldodecanol is particularly preferred.

The nonvolatile solvent may be present in the composition in an amount ranging from about 0.5 wt. % to 25% wt. %, more preferably from 10 wt. % to 20 wt. %, and most preferably from about 13 wt. % to about 17 wt. % of the composition.

In one embodiment, the compositions are free of, or substantially free from, volatile solvents. It has been found that volatile solvents create formulation challenges, resulting in inferior processing conditions and product outcomes. Compositions including material levels of volatile solvents can result in less moldable compositions with inferior feel characteristics.

### Pigment

The compositions comprise a high pigment load. Therefore, the compositions include a pigment level of at least about 35 wt. %, more preferably at least about 40 wt. %, and most preferably at least about 45 wt. % of the composition. The pigment load should also be less than about 60 wt. %, more preferably less than about 55 wt. %, and most preferably less than about 52 wt. % of the composition.

Non-limiting examples of pigments useful in the present compositions include talc, mica, silica, synthetic fluorphlogopite, zinc oxide, manganese violet, ferric ferrocyanide, ultramarine blue, pearl pigments (bismuth oxychloride, guanine, calcium sodium borosilicate, calcium aluminum borosilicate, alumina, polyethylene terephthalate), iron oxide, titanium oxide, titanated mica, calcium carbonate, tar pigments, and other organic pigments. Pigments which are surface treated with silicones, silanes and its derivatives, metal soaps, fluorine compounds and its derivatives, lecithin and its derivatives, amino acids and its salts, etc. can also be used. The pigments can be dispersed in an oily medium such as those disclosed herein among suitable solvents. Pigments can also be pre-dispersed in an oily medium like castor oil, or as dry powders which subsequently are dispersed in a chosen medium.

Preferred pigments and powders include talc, mica, clay, kaolin, zinc oxide, nylon powder, ultramarine, pearl pigments (bismuth oxychloride, guanine), iron oxide, titanium oxide, titanated mica, calcium carbonate, tar pigments, and mixtures thereof. Particularly preferred pigments and powders include mica, titanated mica, and/or mica pearls, such as those available from BASF.

### Wetting Agent

The compositions may also comprise a wetting agent. Suitable wetting agents may include, for example, surfactants which have an HLB from about 5-7, such as sorbitan trioleate, polyglycerol 3 diisostearate, sorbitan sesquioleate, and mixtures thereof. The wetting agent may be present at a level of from about 0.1 wt. % to about 1.5 wt. % of the composition. The wetting agent helps to provide pigment dispersion in compositions with high pigment loads.

### Ultraviolet Absorbing Agent

The compositions herein may include one or more known ultraviolet absorbing agents, preferably at least one compound which absorbs in the UV-B region (wavelength 290 to 320 nanometers) and optionally one or more other compounds which absorb in the UV-A region (wavelength 320 to 400 nanometers). The total amount of UV absorbing agents included within the formulation may be from about 2 wt. % to about 15 wt. %, which amount will determine whether it is a sunscreen or sunblock. As the present compositions are anhydrous, the UV agent is preferably lipophilic.

Suitable UV-A absorbing agents which may be employed include 2-(2′-hydroxy-5′-methylphenyl)benzo-triazole (Tinuvin P); 2-(2′-hydroxy-5′-t-octylphenyl)benzotriazole (Spectra-Sorb UV 5411); 2,4-dihydroxybenzophenone (Uvinul 400); 2-hydroxy-4-methoxybenzophenone (oxybenzone, Spectra-Sorb UV9, Uvinul M-40); 2,2′,4,4′-tetrahydroxybenzophenone (Uvinul D50); 2,2′-dihydroxy-4,4′-dimethoxybenzophenone (Uvinul D49); 2,2′-dihydroxy-4-methoxybenzophenone (dioxybenzone, Spectra-Sorb UV24); 2-ethylhexyl-4-phenyl-benzophenone carbonate (Eusolex 3573); 2-hydroxy-4-methoxy-4′-methylbenzophenone (mexenone, Uvistat 2211); 2-hydroxy-4-(n-octyloxy)benzophenone (octabenzone, SpectraSorb UV531); 4-phenylbenzophenone (Eusolex 3490); and 2-ethylhexyl-2-cyano-3,3′-diphenylacrylate (Uvinul N539); butyl methoxydibenzoyl methane (Parsol 1789), and benzphthalide (Escalol 547).

The UV-A absorbing agent or agents may be present in the final product at from about 0 wt. % to about 10 wt. % of the formulation. The amount will vary according to the particular agent selected and whether the formulation is intended to minimize or permit tanning. Where a UV-A absorbing agent is employed, the preferred UV-A absorbing agent is 2-hydroxy-4-methoxybenzophenone alone or in combination with 2,2′-dihydroxy-4-methoxybenzophenone.

Suitable UV-B absorbing agents include 4-(dimethylamino)benzoic acid, ethyl ester; 4-(dimethylamino)benzoic acid, 2-ethylhexyl ester (Escalol 507); 4-(dimethylamino)benzoic acid, pentyl ester (Escalol 506); glyceryl p-amino-benzoate (Excalol 106); isobutyl p-amino-benzoate (Cycloform); and isopropyl p-amino-benzoate; 2-ethylhexyl methoxy cinnamate (Parsol MCX); phenylbenzimidazole sulfonic acid (Eusolex 232); homomenthyl salicylate, and ethyl hexyl salicylate. The UV-B absorbing agent or agents may be present in the final product at from about 1 wt. % to about 15 wt. % of the formulation. The amount will vary according to the particular agent selected and degree of protection desired in the final product. The preferred UV-B absorbing agent is 4-(dimethylamino)benzoic acid, 2-ethyl-hexyl ester (Escalol 507).

### Additional Optional Ingredients

The compositions herein may optionally contain one or more additional cosmetically acceptable ingredients.

As examples of such additional ingredients, mention may be made of, but not limited to, conventional cosmetic adjuvants selected especially from among fatty substances; organic solvents, ionic or nonionic, hydrophilic or lipophilic thickeners; softeners; humectants; opacifiers; stabilizers; preservative; emollients; silicones; anti-foams; fragrances; preservatives; anionic, cationic, nonionic, zwitterionic or amphoteric surfactants; fillers; polymers; propellants; acidifying or basifying agents; pigments; dyes; cosmetic tanning agents; inorganic photoprotective agents and any other ingredients commonly used in a cosmetic or dermatological composition.

The fatty substances may be an oil. The term “oil” means a compound that is lipophilic liquid at room temperature. Oils that may be mentioned include mineral oils (paraffin); vegetable oils (sweet almond oil, macadamia oil, grapeseed oil or jojoba ester/oil); synthetic oils, for instance perhydrosqualene, fatty alcohols, fatty acids or fatty esters (for instance the C12-C15 alkyl benzoate marketed under the name “Finsolv TN” of “Witconol TN” by Witco, octyl palmitate, isonohyl isononanoate, isopropyl lanolate and triglycerides, including capric/caprylic acid triglycerides), dialkyl carbonate such as dicaprylyl carbonate, oxyethylenated or oxypropylenated fatty esters and ethers; silicone oils (cyclomethicones and polydimethylsiloxanes such as dimethicone) or fluoro oils, and polyalkylenes. Oil containing at least one amide structure such as those disclosed in U.S. Pat. No. 7,357,920, can also be used.

If desired, one or more lipophilic thickeners may be added to the composition in order to adjust the rheological properties of the compositions herein. Lipophilic thickeners that may be mentioned include modified clays such as hectorite and its derivatives, for instance the products marketed under the name “Benton”.

As fillers or polymers which may be added to the composition of the present invention, mention may be made of, but not limited to, natural fibers such as silk, cellulose, and wool; synthetic fibers such as polyamide which is also referred to as “Nylon®”, and rayon; and particles or beads of polymers such as polyamides, polyacrylates, and polymethylsilsesquioxane.

Another optional component of the formulation is one or more c. The use of a film-former improves the wear of the composition and can confer transfer-resistance to the makeup product. Examples of useful film-forming agents include natural waxes, polymers such as polyethylene polymers and copolymers of PVP, dimethicone gum, and resin, such as shellac, polyterpenes, and various silicone resins. A particularly preferred film-former is trimethylsiloxysilicate, used in an amount of from about 0.1-20%.

As described above, an inorganic photoprotective agent may be used together with the lipophilic cosmetic or dermatological active ingredient. An inorganic photoprotective agent used for the present invention may be selected from among pigments and even more preferably nano-pigments (mean size of the primary particles: generally from 5 run to 100 nm and preferably from 10 nm to 50 nm) of treated or untreated metal oxides such as, for example, nano-pigments of titanium oxide (amorphous or crystallized in rutile and/or anatase form), of iron oxide, of zinc oxide, of zirconium oxide or of cerium oxide.

The treated nano-pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen or elastin), alkanolamines, silicon oxides, metal oxides, sodium hexametaphosphate, alumina or glycerol.

Many particular pigments and nano-pigments which can be used are disclosed in U.S. Pat. No. 7,357,920.

### NON-LIMITING EXAMPLES

The compositions illustrated in the following Examples illustrate specific embodiments of the compositions of the present invention but are not intended to be limiting thereof. Other modifications can be undertaken by the skilled artisan without departing from the spirit and scope of this invention. Exemplified embodiments 1-5 provide a moldable, anhydrous, cosmetic composition with a high pigment load. Examples 6-9 are comparative illustrations of formulas which fail to exhibit acceptable cosmetic performance, as discussed in more detail hereinafter.

The compositions according to the following Examples may be prepared according to the following steps: First, mix the synthetic beeswax with each of the microcrystalline waxes and stearyl dimethicone/octadecene in a vessel, which is heated to about 95° C. to form a first phase. Next, in a separate beaker, combine all remaining ingredients, except pigments and particles (mica) at about 95° C. to form a second phase. Next, combine the first and second phases to form a single mixture, maintaining a mixing temperature of about 95° C. Finally, add the pigments and particles to the mixture. Mix until the ingredients form a homogenous composition, then stop mixing and allow the mixture to cool to room temperature.

All exemplified amounts are listed as weight percents and exclude minor materials such as diluents, preservatives, color solutions, imagery ingredients, botanicals, and so forth, unless otherwise specified.

The following are representative of cosmetic compositions of the present invention:

TABLE 1

| Ingredient | 1 | 2 | 3 | 4 | 5 | 6 | 7 | 8 | 9 |
|---|---|---|---|---|---|---|---|---|---|
| Synthetic Beeswax¹ | 5.24 | 5.24 | 6.07 | 6.07 | 5.24 | — | 5.39 | 5.29 | 5.35 |
| Microcrystalline Wax² | 2.84 | 2.84 | 1.70 | 2.50 | 2.84 | 3.00 | — | 2.87 | 2.90 |
| Microcrystalline Wax³ | 2.00 | 1.00 | 2.00 | 2.30 | 2.00 | 2.11 | 2.06 | 2.02 | — |
| Stearyl Dimethicone/Octadecene⁴ | 1.57 | 1.57 | 1.88 | 1.00 | 1.00 | 1.06 | 1.03 | — | 1.02 |
| Octyldodecanol | 16.77 | 16.77 | 16.77 | 16.77 | 15.92 | 16.80 | 16.36 | 16.08 | 16.24 |
| Lecithin | 0.15 | 0.15 | 0.15 | 0.15 | 0.8 | 0.84 | 0.82 | 0.81 | 0.82 |
| Pentaerythrityl Tetraethylhexanoate | 6.08 | 6.08 | 6.08 | 4.08 | 4.00 | 4.22 | 4.11 | 4.04 | 4.08 |
| Trioctyldodeyl Citrate | 3.25 | 3.25 | 3.25 | 3.25 | 3.25 | 3.43 | 3.35 | 3.28 | 3.32 |
| Phenyl Trimethicone | 1.26 | 1.29 | 1.26 | 1.26 | 1.26 | 4.22 | 1.30 | 1.27 | 1.29 |
| Dipentaerythrityl Hexahydroxystearate/ | 1.37 | 1.37 | 1.37 | 1.45 | 2.02 | 2.13 | 2.08 | 2.04 | 2.06 |
| Hexastearate/Hexarosinate | | | | | | | | | |
| Jojoba Ester | 3.15 | 3.15 | 3.15 | 4.00 | 3.15 | 3.32 | 3.24 | 3.18 | 3.21 |
| Butyrospermum Parkii (Shea Butter) | 3.15 | 3.15 | 3.15 | 4.00 | 3.15 | 3.32 | 3.24 | 3.18 | 3.21 |
| Tocopheryl Acetate | 0.10 | 0.10 | 0.10 | 0.10 | 0.10 | 0.10 | 0.10 | 0.10 | 0.10 |
| Caprylyl Glycol | 0.10 | 0.10 | 0.10 | 0.10 | 0.30 | 0.32 | 0.31 | 0.30 | 0.30 |
| Pentaerythrityl Tetra-Di-T-Butyl | 0.15 | 0.15 | 0.15 | 0.15 | 0.15 | 0.16 | 0.15 | 0.15 | 0.15 |
| Hydroxydrocinnamate | | | | | | | | | |
| Silica | 3.52 | 3.52 | 3.52 | 3.52 | 3.80 | 4.01 | 3.91 | 3.84 | 3.84 |
| HDI/Trimehylol Hexyllactone | 1.98 | 1.98 | 2.27 | 2.27 | 2.00 | 2.11 | 2.06 | 2.02 | 2.04 |
| Crosspolymer/Polymethyl Methacrylate | | | | | | | | | |
| Ascorbyl Palmitate | 0.05 | 0.05 | — | — | — | — | — | — | — |
| Pigment | 47.04 | 47.04 | 47.04 | 47.04 | 47.04 | 51.6 | 50.46 | 49.50 | 50.10 |
| Filler⁵ | q.s | q.s | q.s | q.s | q.s | q.s | q.s | q.s | q.s |

¹Synthetic Beeswax #122P, available from Koster Keunen
²Microcrystalline Wax SP 88, available from Strahl & Pitsch
³Multiwax W-835, available from Sonneborn
⁴Dowsil 2503 Cosmetic Wax, available from Dow Corning
⁵The filler may include solvents, fragrances, or other additional optional ingredients disclosed herein

Table 2, below, illustrates comparative cosmetic performance among the examples provided in Table 1. Each exemplary embodiment was observed and evaluated for suitability as a cosmetic composition. First, the compositions were evaluated for processability. It is necessary for the compositions to have good mixing and pourability performance. Second, the product is observed after the processing step, and it is evaluated for drag, payoff, moldability, and sensorial characteristics. As used herein, the term, “drag” means the relative ease with which a product is applied to skin. Sensorially, a product with low drag is perceived to glide onto the skin. In contrast, a product with increased drag will exhibit friction and uneven application to skin. As used herein, the term, “payoff” refers to the color intensity of the cosmetic composition when applied to skin. More intense perceived color intensity correlates with higher relative payoff and less intense perceived color intensity correlates with less relative payoff.

TABLE 2

| EXAMPLE | OBSERVATIONS |
|---|---|
| 1 | Good mixing and pouring performance. The product has |
| | some drag but does not crumble upon application to skin. |
| 2 | Acceptable mixing performance. Formula crumbles |
| | upon application to skin. |
| 3 | Good mixing and pouring performance. Minimal |
| | drag, and cooling sensation on skin. |
| 4 | Good mixing and pouring performance. Minimal |
| | drag, and cooling sensation on skin. |
| 5 | Good mixing and pouring performance. Excellent |
| | glide, payoff, and cooling sensation on skin. |
| 6 | Formula is too thick to mix with an impeller. |
| | The product is too rigid and brittle to mold. It crumbles |
| | when handled. |
| 7 | Formula is very viscous and difficult to mix. It can be |
| | shaped into a ball, but crumbles upon application to skin. |
| 8 | Mixing and pouring performance are acceptable, but |
| | final product drags against skin with uneven payoff. |
| 9 | Mixing and pouring performance are acceptable, but |
| | final product crumbles upon application to skin. |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for forming an anhydrous, moldable, cosmetic composition comprising the steps of:
a. Combining in a vessel, at least four waxes, each of said waxes being characterized by:
i. a first wax having a melting point of between about 60° C. to about 65° C.;
ii. a second wax having a melting point of between about 33° C. to about 46° C.;
iii. a third wax having a melting point of between about 88° C. to about 96° C.;
iv. a fourth wax having a melting point of between about 74° C. to about 99° C.;
b. heating the first vessel to about 95° C. to form a first phase;
c. combining at least one wetting agent and at least one nonvolatile solvent in a second vessel;
d. heating the second vessel to about 95° C. to form a second phase;
e. combining said first and second phase to form a mixture; and
f. adding one or more pigments to said mixture to form said anhydrous, moldable, cosmetic composition.

2. The method according to claim 1, wherein said pigments comprise at least about 35 wt. %, of said cosmetic composition.

3. The method according to claim 1, wherein said cosmetic composition comprises:
a. from about 1 wt. % to about 10 wt. % of said first wax;
b. from about 0.5 wt. % to about 5 wt. % of said second wax;
c. from about 1 wt. % to about 5 wt. % of said third wax; and
d. from about 0.5 wt. % to about 5 wt. % of said fourth wax.

4. The method according to claim 1, wherein said cosmetic composition comprises said first phase at a level of from about 2 wt. % to about 20 wt. % of said cosmetic composition.

5. The method according to claim 1, wherein said cosmetic composition comprises at least about 30 wt. % of said one or more pigments.

6. The method according to claim 1, wherein said cosmetic composition comprises at least about 40 wt. % of said one or more pigments.

7. The method according to claim 1, further comprising one or more UV absorbing agents.

8. The method according to claim 7 wherein said UV absorbing agent is lipophilic.

9. The method according to claim 1, wherein said solvent is nonvolatile.

10. The method according to claim 9, wherein said solvent is selected from the group consisting of silicone-based solvents, ester-based solvents, hydrocarbon-based solvents, and mixtures thereof.

11. The method according to claim 9, wherein said solvent is selected from the group consisting of ethanol, octyldodecanol, isopropanol, and mixtures thereof.

12. The method according to claim 1, where said composition comprises from about 13 wt. % to about 17 wt. % of said solvent.

13. The method according to claim 1, wherein said composition comprises one or more pigments selected from the group consisting of talc, mica, silica, synthetic fluorphlogopite, zinc oxide, manganese violet, ferric ferrocyanide, ultramarine blue, pearl pigments (bismuth oxychloride, guanine, calcium sodium borosilicate, calcium aluminum borosilicate, alumina, polyethylene terephthalate), iron oxide, titanium oxide, titanated mica, calcium carbonate, tar pigments, other organic pigments, and mixtures thereof.

14. The method according to claim 1, wherein said pigment comprises mica.

15. The method according to claim 1, wherein said composition further comprises a film-forming agent.
